(19) United States
(12) Patent Application Publication
(10) Pub. No.: US 2003/0092136 A1
(43) Pub. Date: May 15, 2003

(54) Process for the manufacture of a starch hydrolysate with a high content of dextrose

(76) Inventor: Didier Delobeau, Merville (FR)

Correspondence Address: HENDERSON & STURM LLP, 1213 MIDLAND BUILDING, 206 SIXTH AVENUE, DES MOINES, IA, 50309-4076, US

(21) Appl. No.: 10/113,266

(22) Filed: Apr. 1, 2002

(30) Foreign Application Priority Data
Sep. 26, 2001 (FR) 01 12398

(51) Int. Cl. C12P 19/02

(52) U.S. Cl. 435/105

(57) ABSTRACT

The invention relates to a process for the manufacture of a starch hydrolysate having a high content of dextrose from a liquefied starch milk, comprising the steps consisting in carrying out a saccharification of a liquefied starch milk with the aid of a glucogenic enzyme so as to obtain a syrup having a richness in dextrose of between 75 and 90% by weight, preferably of between 80 and 90% by weight, a first separation by membrane nanofiltration of the saccharified hydrolysate thus obtained so as to recover a nanofiltration retentate and a nanofiltration permeate, this permeate constituting a starch hydrolysate having a content of dextrose greater than 95% by weight, preferably greater than 96% by weight, and more preferably still greater than 97% by weight, and a second separation by membrane nanofiltration of the preceding nanofiltration permeate so as to obtain the said starch hydrolysate having a high content of dextrose.

The subject of the invention is a process for the manufacture of a starch hydrolysate with a high content of dextrose.

The subject of the invention is also a process for the manufacture of sorbitol from a starch hydrolysate with a high content of dextrose, obtained using the process in accordance with the invention.

The expression “starch hydrolysate with a high content of dextrose” is understood to mean a starch hydrolysate with a content of dextrose greater than 99.5% by weight.

It is known to manufacture starch hydrolysates for which the value of the dextrose equivalent (reducing power expressed as glucose over the dry matter content, hereinafter DE) is 2 to 98 and which, based on this value, may contain up to 96% by weight of true dextrose.

These various qualities of starch hydrolysates are obtained through the choice of the conditions of the hydrolysis of the starting starch.

The nature of the hydrolysis, that is to say whether it is acid or enzymatic, also plays a role.

Starch hydrolysates rich in dextrose, although having numerous fields of application, serve mainly as raw material for the manufacture of crystallized dextrose or as substrate for the manufacture of fructose by isomerization.

For these two applications, the highest possible conversion is sought, that is to say the highest possible content of dextrose, with a minimum of impurities.

In order to obtain these contents of true dextrose, several strategies have been proposed in order to either improve the conversion of the starch by limiting the formation of coproducts, or to improve the efficiency of the dextrose/coproduct (oligosaccharides and polysaccharides) separation.

Thus, according to the first strategy, a process consists in carrying out the steps of liquefaction and saccharification at very low dry matter contents (of the order of 5 to 10%). However, even at such low levels of dry matter, the richness in true dextrose does not exceed 95 to 97% by weight. In addition, such a process is not at all economically viable because of the energy required for the evaporation of the water.

Another process consists in carrying out the saccharification in the presence of an enzyme which hydrolyses the 1-6 bonds of starch, but even in this case, the content of dextrose only reaches a maximum of 96 to 97% by weight.

According to the second strategy, a first process consists in separating, in a manner known per se, the dextrose and the oligosaccharides and polysaccharides by passing the hydrolysate over a column of a molecular sieve such as a cationic resin. In such a process, the aqueous starch hydrolysate which has been previously subjected to a pretreatment such as a concentration, a filtration and/or a decoloration, is adsorbed onto the column, the coproducts (the polysaccharides and a portion of the oligosaccharides) being present in the raffinate excluded from the sieve.

The dextrose is then desorbed by elution with water, the latter then being partially or completely removed to form a concentrated solution of dextrose or crystallized dextrose.

Another process, based on the same principle as that mentioned above, consists in separating the dextrose and the oligosaccharides and polysaccharides by passing the starch hydrolysate over tangential filtration membranes. Such a process is described in the documents FR-A-2,762,616 and U.S. Pat. No. 5,869,297.

The latter process effectively allows the production of a starch hydrolysate having a high content of true dextrose greater than 98-99% by weight, but the yields obtained are unfortunately too low (of the order of 20 to 25%) to justify such processes from the industrial and economic points of view.

To try to correct the disadvantages of these two strategies, solutions have been proposed, which consist in controlling both the conditions for saccharification and those for separation of the dextrose from its coproducts.

In patent application WO 99/27124, a process is described for saccharifying a solution of liquefied starch, comprising a saccharification step during which one or more enzymatic saccharification steps occur, and comprising the steps which consist in one or more steps of membrane separation at high temperature, and recirculation of the saccharification enzyme, in which process the membrane separation steps are carried out as an integral part of the saccharification step.

These numerous separation steps involve ultrafiltration or nanofiltration membranes because it is necessary to trap the enzyme in order to cause it to recirculate.

For the preparation of very pure dextrose, at more than 99% by weight, it is a nanofiltration membrane that has to be used, in which case it is advantageous not to saccharify at more than 92% by weight of dextrose because there are fewer products of reversion.

The separation on nanofiltration membranes is carried out at high temperature, greater than 60° C., and preferably between 63 and 80° C.

Patent application EP No. 452,238 describes a process for producing dextrose having a richness greater than 99% by weight, consisting in carrying out the nanofiltration of a syrup of 95 to 96% by weight of dextrose, but here again at a temperature of about 60° C.

U.S. Pat. No. 4,594,322 describes and exemplifies a process for producing a solution of dextrose consisting in saccharifying a starch liquefied with a soluble or insoluble amyloglucosidase in a single reactor or a reactor consisting of several zones, for which syrup it is said that the richness may exceed 99% by weight of dextrose by appropriately varying the different variables of the process.

The process should nevertheless follow a step of membrane filtration and always requires the recycling of the enzyme and of the retentate undergoing saccharification.

The final richness in dextrose is however entirely relative because there is obtained only a syrup containing 98% by weight of dextrose from a hydrolysate containing 83.4% by weight of dextrose with the aid of a filtration membrane having a cut-off of 500 Daltons, at a temperature of 60° C., or a syrup containing 93.6% by weight of dextrose from a syrup containing 90.5% by weight of dextrose, with the aid, this time, of a filtration membrane having a cut-off of 10 000 Daltons and the enzyme and the retentate undergoing saccharification are recycled.

This need to recirculate the enzyme is again illustrated in patent U.S. Pat. No. 3,720,583 which describes the production of dextrose solutions by saccharification of a liquefied starch by passing over membranes, and recycling the enzyme and the retentate undergoing saccharification.

The temperatures for saccharification and passing over membranes should however be less than 50° C. in order to obtain the longest half-life of the enzyme.

However, even if two types of membranes with cut-offs of 1 000 and 50 000 Daltons are used, they only make it possible to obtain syrups containing 98.9% by weight of dextrose and syrups containing 93 to 97% by weight of dextrose, respectively.

A first object of the present invention is therefore to provide a process for the manufacture of a starch hydrolysate having a high content of dextrose which makes up for the limits and/or the disadvantages of the processes known in the prior art.

Another object of the present invention is to provide a process for the manufacture of a starch hydrolysate having a high content of dextrose, greater than 99.5% by weight, without the need to recirculate the saccharification enzyme, or to use membrane separation steps at high temperature.

This thus leads to the proposal of a simple and economically efficient process which makes it possible to obtain, with very satisfactory yields, hydrolysates with an equally high content of true dextrose.

To this effect, the invention provides a process for the manufacture of a starch hydrolysate having a high content of dextrose from a liquefied starch milk, comprising the steps consisting in carrying out:

a) a saccharification of the liquefied starch milk with the aid of a glucogenic enzyme so as to obtain a syrup having a richness in dextrose of between 75 and 90% by weight, preferably of between 80 and 90% by weight,

b) a first separation by membrane nanofiltration of the saccharified hydrolysate thus obtained so as to recover a nanofiltration retentate and a nanofiltration permeate, this permeate constituting a starch hydrolysate having a content of dextrose greater than 95% by weight, preferably greater than 96% by weight, and more preferably still greater than 97% by weight,

c) a second separation by membrane nanofiltration of the preceding nanofiltration permeate so as to obtain the said starch hydrolysate having a high content of dextrose.

The first step of the process in accordance with the invention consists in carrying out a saccharification of a liquefied starch milk with the aid of a glucogenic enzyme so as to obtain a syrup having a richness in dextrose of between 75 and 90% by weight, preferably of between 80 and 90% by weight.

The general outline for carrying out the starch liquefaction step is known by persons skilled in the art and may consist in a controlled hydrolysis of the starch milk in order to obtain a liquefied starch milk having a low conversion level, for example according to the teaching of U.S. Pat. No. 6,126,754 of which the Applicant company is the assignee.

As for the actual saccharification step, after numerous investigations, the Applicant company has observed that in a process for the manufacture of a starch hydrolysate having a high content of dextrose using a membrane separation step, the richness of the permeate in dextrose was better if the saccharified starch hydrolysate to be separated had a richness in dextrose of between 75 and 90% by weight, preferably of between 80 and 90% by weight.

During this saccharification step, a liquefied starch milk is therefore subjected to the action of a glucogenic enzyme, selected in particular from the group consisting of amyloglucosidase, glucoamylase or any other glucogenic enzyme.

To avoid reversion reactions and the formation in particular of disaccharides (maltose, isomaltose) by repolymerization of the dextrose, the saccharification step is carried out under conditions and in a manner known per se, for at least 24 hours.

Indeed, the preferred substrate for the glucogenic enzymes has a high molecular weight, and the α-1,4 bonds of the starch are hydrolysed much more rapidly than the α-1,6 bonds. Consequently, at the beginning of saccharification, the large molecules and the α-1,4 bonds being predominant, the production of dextrose is extremely rapid whereas the production of the reversion products is very slow because of the low concentration of dextrose in the reaction medium.

As the saccharification progresses, the small molecules and the α-1,6 bonds becoming predominant, the level of production of the dextrose decreases gradually whereas the production of the reversion products (highly branched oligosaccharides) accelerates.

To correct this phenomenon, it may be advantageous to combine the glucogenic enzyme with an enzyme specifically hydrolysing the α-1,6 bonds of the starch. This addition of a debranching enzyme makes it possible, on the one hand, to accelerate the hydrolysis reactions without simultaneously accelerating the reversion reactions, and, on the other hand, to reduce the quantity of highly branched oligosaccharides which are normally resistant to the action of the glucogenic enzyme. Preferably, the debranching enzyme is isoamylase or pullulanase.

The quantities and the conditions for the action of the various enzymes used in the process in accordance with the invention are selected from the following:

amyloglucosidase: 4 000 to 400 000 international units per kilogram of dry substrate, temperature from 50° C. to 60° C., maximum duration of action 24 hours, pH from 4 to 6;

pullulanase: 150 to 15 000 ABM units.

The enzymes used may be of bacterial or fungal origin.

The second step of the process in accordance with the invention consists in carrying out a first separation by membrane nanofiltration of the saccharified hydrolysate thus obtained so as to recover a nanofiltration retentate and a nanofiltration permeate, this permeate constituting a starch hydrolysate having a content of dextrose greater than 95% by weight, preferably greater than 96% by weight, and more preferably still greater than 97% by weight.

After numerous investigations, the Applicant company has thus had the merit of showing that in a process using two separations by membrane nanofiltration, to ensure in the end a richness of the starch hydrolysate in dextrose at more than 99.5% by weight, it was necessary to manage the conditions for carrying out this first nanofiltration step so that the dextrose content of the first permeate is greater than 95% by weight, preferably greater than 96% by weight, and more preferably still greater than 97%.

It is known, moreover, by persons skilled in the art that the disadvantage of purification processes by nanofiltration is the lack of stability of the results over time, the ageing of the membranes leading to a reduction in the richness of the permeate over time.

However, surprisingly and unexpectedly, the Applicant company has shown that carrying out the nanofiltration under the conditions mentioned above also makes it possible to maintain over time a high richness in dextrose in the permeate from the second nanofiltration, despite the inherent ageing of the said membranes.

As will be exemplified below, the results obtained with membranes which have had more than 3 months of continuous operation confirm this threshold value at 95% by weight of dextrose to be complied with for the syrup nanofiltered a first time in order to ensure, after the second nanofiltration, a final richness in dextrose of the starch hydrolysate at a value greater than 99.5%.

According to a preferred embodiment, the first membrane separation is then carried out under temperature conditions of between 30° C. and 60° C., preferably of between 40° C. and 50° C. and pressure conditions of between 15 and 35 bar, and preferably of between 20 and 30 bar.

The nanofiltration membrane advantageously used in the process in accordance with the invention is of the NF40 type marketed by the company FILMTEC or of the DESAL 5 DL 3840 type marketed by the company DESALINATION SYSTEMS.

According to a particular embodiment of the invention, this first retentate is also completely or partially recycled at the head of the first nanofiltration, which makes it possible to increase the recovery yield of dextrose in the first nanofiltration permeate.

The third step of the process in accordance with the invention consists in carrying out a second separation by membrane nanofiltration of the preceding nanofiltration permeate so as to obtain the said starch hydrolysate containing a high content of dextrose.

Contrary to the teaching of the state of the art, which recommends at this stage of the membrane separation process to complete the saccharification of the retentate in order to enrich in dextrose the permeate which will finally constitute the syrup with a high dextrose content, the Applicant company has shown that this result could be obtained more advantageously by the second nanofiltration of the permeate from this first membrane separation.

According to a preferred embodiment, the membrane separation is carried out under the same operating conditions as those used for the first nanofiltration step.

The permeate from this second nanofiltration step obtained in accordance with the process of the invention contains more than 99.5% by weight of richness in dextrose.

The retentate from this second nanofiltration step which contains more than 95% by weight of dextrose may be completely or partially recycled at the head of the first nanofiltration or of the second nanofiltration, or both, or may be advantageously upgraded in the context of the crystallization of the dextrose in order to obtain a crystallized dextrose monohydrate of high quality.

The dextrose derived from the permeate from this last nanofiltration may, for its part, be easily catalytically hydrogenated.

The hydrogenation of such a dextrose is carried out in accordance with the rules of the art which lead, for example, to the production of sorbitol from glucose.

It is possible to use for this step both ruthenium-based catalysts and Raney nickel catalysts.

The use of Raney nickel catalysts which are less expensive is however preferred.

In practice, 1 to 10% by weight of catalyst is used relative to the dry matter content of the hydrolysate subjected to hydrogenation.

The hydrogenation is preferably carried out on a hydrolysate whose dry matter content is between 15 and 50%, in practice in the region of 30 to 45%, at a hydrogen pressure of between 20 and 200 bar.

It may be carried out continuously or batchwise.

When the procedure is carried out batchwise, the hydrogen pressure used is generally between 30 and 60 bar and the temperature at which the hydrogenation is carried out is between 100 and 150° C.

Care is also taken to maintain the pH of the hydrogenation medium by addition of sodium hydroxide or of sodium carbonate, for example, but without exceeding a pH of 9.0.

This manner of proceeding makes it possible to avoid the appearance of products of cracking or of isomerization.

The reaction is stopped when the reducing sugar content of the reaction medium is less than 1%, preferably still less than 0.5% and more preferably less than 0.1%. After cooling of the reaction medium, the catalyst is removed by filtration and the sorbitol thus obtained is demineralized on cationic and anionic resins.

At this stage, the syrups contain at least 98% of sorbitol and it is easy to solidify the latter by crystallization after concentration and cooling of the solutions.

Other characteristics and advantages of the invention will appear clearly on reading the examples which follow. They are however given here only by way of illustration and without limitation.

### EXAMPLE 1

The determination of the threshold value of richness in dextrose of the feed syrup which has to be subjected to separation by membrane nanofiltration in order to obtain a syrup containing more than 99.5% of dextrose is carried out with 5 syrups containing a dextrose content varying from 93.1 to 97.1% by weight.

These 5 hydrolysates are subjected to a continuous nanofiltration with membranes which have had more than 3 months of continuous operation, under the following operating conditions:

Membrane DESAL 5 DL

Temperature: 45° C.

Pressure: 25 bar

The following Table I presents the richness in dextrose of each of the five permeates obtained.

TABLE I

| % of dextrose in the | % dextrose in the |
|---|---|
| feed syrup | permeate |
| 93.1 | 98.9 |
| 94.3 | 99.1 |
| 94.8 | 99.4 |
| 96.3 | 99.5 |
| 97.1 | 99.6 |

The results show that effectively, in order to ensure over time a richness in dextrose at more than 99.5% by weight, it is necessary to nanofilter a solution in dextrose greater than 95% by weight, preferably greater than 96% by weight and more preferably still greater than 97% by weight.

### EXAMPLE 2

A starch milk is liquefied in a conventional manner using 0.5 per thousand of THERMAMYL 120L (α-amylase marketed by the company NOVO) to a DE of 6.5.

The reaction medium is then heated for a few seconds at 140° C. so as to inhibit the α-amylase.

The saccharification of the hydrolysate containing 26.7% of dry matter is then carried out, in a manner known per se, in the presence of 0.8 per thousand of amyloglucosidase G990 marketed by the company ABM (temperature: 60° C., pH=4.5).

After 22 hours of saccharification, a hydrolysate is obtained which has the following carbohydrate spectrum:

glucose: 86.3%

DP2: 4.5%

DP3 and higher: 9.2%

it being understood that the abbreviation “DP” means degree of polymerization.

This hydrolysate is subjected to a continuous nanofiltration under the following operating conditions:

Membrane DESAL 5 DL

Temperature: 45° C.

Pressure: 25 bar

The retentate is partially recycled at the head of this first nanofiltration and the permeate then obtained subjected to a second nanofiltration, under the same operating conditions.

The following Table II presents the characteristics of the different retentates and permeates of the first and second nanofiltration, the values for feeding the first nanofiltration taking into account the dilution factor provided by the recycling of the first retentate.

In accordance with the results of Example 1, the operating conditions make it possible to obtain, at the end of the first nanofiltration, a syrup having a richness in dextrose of 97.1%.

TABLE II

| | Feeding | Recycling | Purge | | Feeding | | |
|---|---|---|---|---|---|---|---|
| | 1st | Retentate | Retentate | Permeate | 2nd | Retentate | Permeate |
| | nanofiltration | No. 1 | No. 1 | No.1 | nanofiltration | No. 2 | No. 2 |
| Composition | | | | | | | |
| % DP1 | 78.8 | 59.6 | 59.6 | 97.1 | 97.1 | 95.2 | 99.6 |
| % DP2 | 13.7 | 25.4 | 25.4 | 2.6 | 2.6 | 4.4 | 0.4 |
| % DP3 and + | 7.5 | 15 | 15 | 0.3 | 0.3 | 0.4 | 0 |
| % dry matter | 29 | 38 | 38 | 24 | 24 | 31 | 19 |
| Throughput | 6 | 1.3 | 0.9 | 3.8 | 7.6 | 3.2 | 4.4 |
| (kg/h · m² of | | | | | | | |
| membrane) | | | | | | | |

The process in accordance with the invention thus makes it possible to obtain a hydrolysate with a high content of dextrose using these two steps of nanofiltration in series, with recycling of the first retentate at the head of the first nanofiltration.

This dextrose can then be advantageously used in all fields of applications requiring the availability of a highly concentrated dextrose, i.e. for the manufacture of crystallized dextrose or as substrate for the manufacture of fructose by isomerization.

### EXAMPLE 3

The second permeate of Example 2, purified and then concentrated to a dry matter content of 45%, is subjected to a catalytic hydrogenation in the presence of 5% by weight of Raney nickel relative to the dry matter content.

The operating conditions are the following:

temperature: 130° C.

pressure: 50 bar

duration: 2 hours

The hydrogenation is stopped when the reducing sugar content of the reaction medium is less than 600 ppm.

After cooling the reaction medium, the catalyst is removed by filtration and then the syrup obtained is demineralized and it is finally concentrated to 70% of dry matter content.

The composition of the syrup thus obtained on a dry matter basis is the following:

sorbitol: 98.8%

mannitol: 0.6%

iditol and cracking products: 0.3%

1. Process for the manufacture of a starch hydrolysate having a high content of dextrose from a liquefied starch milk, comprising the steps consisting in carrying out:
a) a saccharification of a liquefied starch milk with the aid of a glucogenic enzyme so as to obtain a syrup having a richness in dextrose of between 75 and 90% by weight, preferably of between 80 and 90% by weight, p1 b) a first separation by membrane nanofiltration of the saccharified hydrolysate thus obtained so as to recover a nanofiltration retentate and a nanofiltration permeate, this permeate constituting a starch hydrolysate having a content of dextrose greater than 95% by weight, preferably greater than 96% by weight, and more preferably still greater than 97% by weight, p1 c) a second separation by membrane nanofiltration of the preceding nanofiltration permeate so as to obtain the said starch hydrolysate having a high content of dextrose.

2. Process according to claim 1, wherein step (a) of saccharification is carried out for a maximum of 24 hours.

3. Process according to claim 1, wherein the first nanofiltration retentate is completely or partially recycled at the head of the said step of separation by membrane nanofiltration.

4. Process according to claim 1, wherein the second nanofiltration retentate is completely or partially recycled at the head of the first nanofiltration or of the second nanofiltration, or both, or may be advantageously upgraded in the context of the crystallization of the dextrose.

5. Process for the manufacture of sorbitol by hydrogenation of a starch hydrolysate having a high content of dextrose, wherein the said hydrolysate is obtained using the process in accordance with claim 1.
